(19) United States
(12) Patent Application Publication
(10) Pub. No.: US 2004/0191837 A1
(43) Pub. Date: Sep. 30, 2004

(54) Method for detecting cells

(76) Inventors: Stuart Harbron, Berkhamsted Hertfordshire (GB); David Bertram Whitehouse, Kings Langley Hertfordshire (GB); David Ross Williams, Lincoln (GB); Kathleen Ann Grant, Hitchin (GB)

Correspondence Address: Bradley N Ruben, Suite 5A, 463 First Street, Hoboken, NJ, 07030-1859, US

(21) Appl. No.: 10/467,997

(22) PCT Filed: Feb. 14, 2002
(86) PCT No.: PCT/GB02/00642

(30) Foreign Application Priority Data
Feb. 14, 2001 (GB) 0103611.0

(51) Int. Cl. G01N 33/53; G01N 33/567

(52) U.S. Cl. 435/7.2

(57) ABSTRACT

A method for detecting the presence of cells or other target biological entities in a fluid sample, which method comprises: a) contacting the sample with a specific binding partner having (i) an electrophoretic, or zeta potential, label or (ii) a fluorescence label; b) allowing the specific binding partner to bind to any said cell or other target biological entity present in the fluid sample; c) in an electric field measuring the value of the velocity, displacement, zeta potential or electrophoretic mobility of any said cell or other target biological entity present in the fluid sample and that is bound to the specific binding partner; and d) observing the value obtained in step c) as indicative of the presence of a said cell or other target biological entity in the fluid samples.

### FIELD OF THE INVENTION

The present invention relates to methods and apparatus primarily for the detection of the presence of cells in a fluid but which may be used for detecting other biological entities. It is particularly applicable, but not necessarily limited, to identifying cells, which may, for example, be micro-organisms such as microbial pathogens and may be used for species, variant or strain determination

### BACKGROUND OF THE INVENTION

There are many applications in which it is important to be able to detect the presence of a specific cell such as a micro-organism. For example, in combating viral or bacterial infections, it is necessary to be able to identify the micro-organism responsible. In certain military situations it is important to know quickly if there is an infective agent in the environment and if so, what it is. In this context the term micro-organism has a broad meaning. It encompasses bacteria, viruses and fungi as well as an individual animal cell, for example a blood cell, or a plant cell, for example an alga.

In both these examples speed of analysis is extremely important. For instance, in diagnosis of a medical problem the medical practitioner needs to know what organism is causing the symptoms within hours rather than days. The most appropriate treatment can then be started straight away, giving the patient the best chance of a speedy recovery. In some cases, such as meningitis, rapid and accurate diagnosis is a matter of life and death.

Under present arrangements samples are usually sent to a pathology laboratory for culture and subsequent identification. By the very nature of the procedure this takes days rather than hours. There may be more than one organism present which requires a number of different cultures in different media.

The use of electrophoretic mobility or zeta potential measurements to identify micro-organisms has been disclosed in our earlier UK patent GB 2,348,504, the entire text of which is hereby imported by reference. Whilst this method is a major improvement over the prior art it does not always provide the sensitivity to differentiate uniquely between all micro-organisms. Whilst it can usually be relied upon to provide valuable information on what class of organism is present and is more reliable than the prior art methods that preceded it (and which is documented in the introduction of this patent), they cannot always be used to identify which strain of organism is present.

By way of a little background explanation, electrophoretic mobility is the velocity a particle has per unit of electrical field strength, and typically has the units of μm per second per Volt per cm or μm/s/V/cm. This value can either be measured under micro-electrophoresis conditions as described by Moyer (J Bacteriol (1936) 31:531-546) or by using a commercially available instrument, such as the Malvern Zetasizer 2000.

Zeta potential, ζ, is derived from electrophoretic mobility by the equation:

ζ=μη/ε

where u is the electrophoretic mobility, η is the viscosity and ε is the dielectric constant. Van der Wal et al (Langmuir (1997) 13:165-171), however show that further factors need to be introduced into this equation to give a true conversion of electrophoretic mobility into zeta potential.

It will be appreciated that in solution the velocity and hence distance travelled by a micro-organism under the influence of an applied electrical field will be proportional to the electrophoretic mobility. It follows therefore that it is not strictly necessary to compute the electrophoretic mobility or zeta potential in a series of experiments where the dielectric constant and viscosity of the various solutions are substantially constant. It is therefore quite sufficient to determine the velocity or distance travelled per unit time providing, as stated above, the experimental conditions remain substantially constant. This approach can simplify the computations significantly if image analysis is used.

Electrophoretic mobility measurement has been used in the past in methods for detecting antibodies (U.S. Pat. No. 3,984,533), in methods for carrying out general cell examination (U.S. Pat. No. 4,783,419) and in methods for determining analytes in solution (U.S. Pat. No. 5,686,252) in which an immunological (antigen-antibody) binding reaction reduces electrophoretic mobility of antigen-labelled particles or cells in solution. However, the prior art has not previously proposed methods for detecting specific cells using immunological binding reactions and measurement of electrophoretic mobility. Furthermore we have found that prior art methods generally do not provide sufficiently clear measurements to give reliable results.

### SUMMARY OF THE INVENTION

According to a first aspect of the present invention there is provided a method for detecting the presence of cells or other target biological entities in a fluid sample, which method comprises:

a) contacting the sample with a specific binding partner having (i) an electrophoretic, or zeta potential, label or (ii) a fluorescence label;

b) allowing the specific binding partner to bind to any said cell or other target biological entity present in the fluid sample;

c) in an electric field measuring the value of the velocity, displacement, zeta potential or electrophoretic mobility of any said cell or other target biological entity present in the fluid sample and that is bound to the specific binding partner; and

d) observing the value obtained in step c) as indicative of the presence of a said cell or other target biological entity in the fluid samples.

The electrophoretic or zeta potential label may be any molecule having a charged group. The label is suitably a polyamino acid such as poly-lysine or poly-glutamate, a charged polysaccharide, such as chitin, a polynucleotide such as DNA or RNA, a charged polymer and the like.

As for the specific binding partner, this may be any moiety that binds specifically to a group on the surface of a particular cell, or to a group on the surface of a related cell. The specific binding partner is preferably selected from the group consisting of: an antibody; a bacteriophage; a ligand for a receptor on the cell's surface; or an antibiotic.

By the provision of a selected zeta potential label on the specific binding partner, the user has the ability to determine the nature of the change that will be observed and which may, for example, be an increase in final velocity of the cells that bind the specific binding partner, or may alter their direction of travel. The use of a positive zeta potential label will result in cells acquiring a velocity in the opposite direction to that which they had before the binding reaction.

In a particularly preferred embodiment of the method of the invention, the sample is first divided into two or more aliquots and then each aliquot is contacted with a different specific binding partner, allowing the binding partners to bind to any cells present in the respective aliquot, and measuring the velocity, displacement, zeta potential or electrophoretic mobility of each aliquot. The pattern of changes in the values of the measured velocity, displacement, zeta potential or electrophoretic mobility with each of the two or more specific binding partners, forms a profile or fingerprint for the particular cell or cells present in the samples.

As noted above, the cell may be an animal or plant cell, a bacterium or a fungal cell. Particularly preferably the method of the present invention is a method for the detection, speciation or determination of a micro-organism present in a sample. It is highly effective and rapid in contrast to prior methods.

A further advantage of the present invention is that the assay may be homogeneous, requiring no separation step. The reagents mixed in the liquid phase need no secondary handling or washing step for the measurements to be able to be taken.

In one preferred embodiment the specific binding partner for the target cell is labelled with a fluorescent moiety.

According to another preferred embodiment of the invention, following or simultaneous with the binding step with the zeta labelled specific binding partner, there is a second binding step in which a second specific binding partner having a fluorescent moiety attached thereto binds to the cell at a specific site that differs from the site of the binding of the zeta labelled specific binding partner.

An appropriate electric field is then applied to the suspension. This will cause the zeta labelled particles in the mixture to move with a characteristic velocity. However, the fluorescent moiety will move only if the link with the zeta moiety has occurred. Thus movement characteristic of the applied field or force is only detected when a binding has formed between the target cell and both the zeta and fluorescence moieties.

In a further embodiment, a plurality of specific binding partners having different zeta moieties are utilised, each specific binding partner having a different specificity for a different target cell, for example where different target cells are different species of bacteria. This embodiment makes it possible to detect multiple target cells in a sample simultaneously. Alternatively or additionally, a plurality of specific binding partners may be used having different fluorescence moieties.

Thus if n different specific binding partners having n different zeta moieties are used in combination with m different specific binding partners having m different fluorescence moieties, a total of m×n different target cells may be determined simultaneously.

If a particular target cell is present, a detectable complex will be formed that has a velocity characteristic of the applied field and the zeta label used.

For a multiplex application, multiple velocity information will be obtained that is characteristic of each of the zeta labels used. Additionally or alternatively, where multiple different fluorescent labels have been used, fluorescent light will be emitted at different wavelengths and this may be used to distinguish between the species. A number of fluorescent dyes are suitable for this application. The criteria for choice is that the excitation wavelength should be within about 25-50 nm of the wavelength of the laser used, and that it may be attached to a specific binding partner without deleteriously affecting the binding process. Dyes that are contemplated for this application include: acridine, AMCA, Alexa fluor 488 and 546, Bodipy labels, cascade blue, the Cy range of labels, dabcyl, edans, eosin, erythrosine, FITC, fluorescein, 6-Fam, Tet, Joe, Hex, Lucifer yellow, NBD, nuclear fast red, nuclear yellow, Oregon green, propidium iodide, rhodamine 6G, rhodamine green, rhodamine red, rhodol green, Tamra, Rox, Texas red, thiazine red R, and true blue.

In a further embodiment, the light source may be polychromatic thus allowing a broader choice of fluorescence moieties, which can be detected and analysed simultaneously.

### BRIEF DESCRIPTION OF THE DRAWINGS

A preferred embodiment of the present invention will now be more particularly described by way of example and with reference to the accompanying drawing, wherein:

FIG. 1 is a graph of zeta potential measurements from cell-sized latex particles with different zeta potential ‘labels’ showing how readily they may be resolved between in a common vessel, to facilitate multiplexing analysis.

### DETAILED DESCRIPTION OF THE PREFERRED EMBODIMENT

In the preferred embodiment of the present invention, the velocity, displacement, zeta potential or electrophoretic mobility of any cells present in a sample is first measured. The method of measurement for any of these criteria is suitably as set out in our earlier UK patent GB 2,348,504.

Then a binding agent is introduced, and after a predetermined time sufficient to allow binding of the binding agent to cells present in the mixture, the velocity, displacement, zeta potential or electrophoretic mobility of the solution is measured a second time. A change in the velocity, displacement, zeta potential or electrophoretic mobility indicates the presence of the cell.

In a preferred example, the binding agent is an antibody labelled with an electrophoresis or zeta potential label that has affinity for a group on the cell of interest. In other embodiments, the binding agent may be a bacteriophage or an antibiotic.

The covalent attachment of the label may be achieved by a number of well-known methods using, for example a wide range of heterobifunctional reagents. For example, the method of Carlsson et al (Biochem J (1978) 173:723-737) may be used: the label is reacted with 3-[(2)-pyridyldithio]propionic acid N-hydroxysuccinimide ester (SPDP) to give a 2-pyridyl disulphide-activated label. This is mixed with an IgG antibody, and a disulphide exchange reaction yields a labelled antibody conjugate. Other approaches for labelling the antibody will be apparent to one skilled in the art. Other methods are described by Tijssen in ‘Practice and theory of enzyme immunoassays’, published by Elsevier, 1985, pages 221 and following.

The concentration of the specific binding agent used is chosen so that the amount of light scattered by the agent is at least 100 times less than the amount of light scattered by the bacterium, if present. Because the binding agent is small in relation to the size of the cell, the amount of light scattered by the binding agent is insignificant.

### EXAMPLE1

### Detection of Escherichia coli using prior art methods

The bacterial strains used in this study were Escherichia coli W3110, Bacillus cereus, Enterococcus faecalis, Pseudomonas aeruginosa, Staphylocuccus saprophyticus, Proteus mirabilis and Klebsiella aerogenes. Cultures were grown in nutrient broth at 37° C. with shaking until the optical density at 600 nm was 0.3. An aliquot of each culture (100μ) was added to 10 ml of 10 mM phosphate buffer pH7.0. The buffer solution had been filtered through a 0.2 μm filter prior to use to remove small particles that may interfere with subsequent electrophoretic measurements. E coli-specific antibodies were used at a dilution of 1 in 20. Electrophoretic mobilities and the derived zeta potentials were obtained by analysing the solutions in a Malvem Zetasizer 2000.

The results are shown in the following Table:

TABLE 1
Change in zeta potential (mV) following addition of antibody

| Strain | Control | + Antibody | Change |
|---|---|---|---|
| E coli | −41.2 | −18.7 | 22.5 |
| B cereus | −32.1 | −32.1 | 0 |
| Ent faecalis | −27.6 | −27.9 | 0.3 |
| Ps aeruginosa | −25.6 | −23.4 | 2.7 |
| Staph saprphyticus | −43.3 | −42.8 | 0.5 |
| P mirabilis | −41.2 | −40.5 | 0.7 |
| Kleb aerogenes | −43.0 | −43.7 | 0.7 |

The data show that where the antibody binds to the cell (E coli, and Ps aeruginosa), the zeta potential becomes less negative. The data also show that a pure culture of E coli may be distinguished from the other bacteria tested. However, in a mixture of bacteria, distinguishing between multiple peaks is more difficult, and it is harder to obtain an unambiguous result. By using a zeta potential label that gives the bacteria a zetapotential more negative than about −50 mV, or a positive value, the discrimination becomes much easier.

### EXAMPLE 2

### Zeta Potential Labels

TABLE 2
lists a number of compounds that may be used as zeta potential
labels.

| Compound | Buffer Conditions | Zeta potential (mV) |
|---|---|---|
| Amino dextran | pH 7.0 | −7.4 |
| 2,3-diamino-2,3-deoxy | pH 7.0 | −21.3 |
| cylcodextrin | | |
| N,0-ethylamine chitosan | 1M acetic acid | +29 |
| Chitosan | 1M acetic acid | +66 |
| Poly-L-arginine | pH 9.2 | +78 |
| Poly-D-lysine | pH 9.2 | +68 |

Compounds were dissolved to a final concentration of 0.5%, and the zeta potential measured using the Malvern Zetasizer 3000.

### EXAMPLE 3

### Multiplexing Zeta Potential Labels

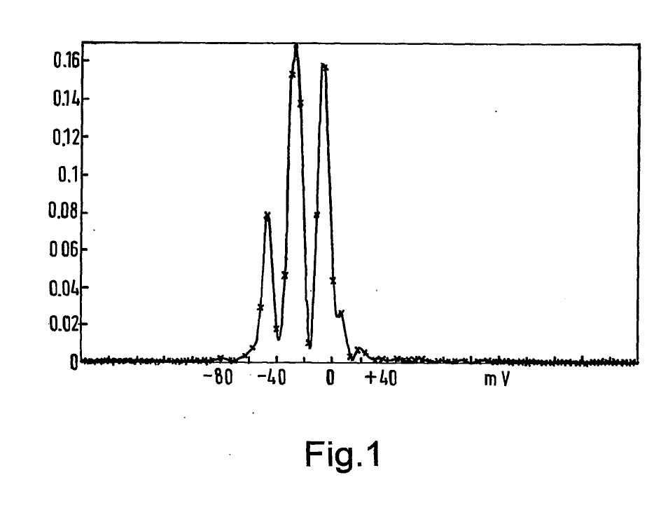

FIG. 1 illustrates the results from simultaneous detection of three differently zeta potential labelled 300 nm diameter latex beads; namely—Carboxy modified, -carboxy and -sulphate “labelled”, suspended together in 10 mM Bis Tris buffer, pH 9.0, measured in a zeta potential reader using laser Doppler anemometry and clearly demonstrate that such small cell-sized entities can be readily resolved between in the same vessel using their different zeta potential profiles arising from their different zeta potential labels.

1. A method for detecting the presence of cells or other target biological entities in a fluid sample, which method comprises:
a) contacting the sample with a specific binding partner having (i) an electrophoretic, or zeta potential, label or (ii) a fluorescence label;
b) allowing the specific binding partner to bind to any said cell or other target biological entity present in the fluid sample;
c) in an electric field measuring the value of the velocity, displacement, zeta potential or electrophoretic mobility of any said cell or other target biological entity present in the fluid sample and that is bound to the specific binding partner; and
d) observing the value obtained in step c) as indicative of the presence of a said cell or other target biological entity in the fluid samples.

2. A method as claimed in claim 1, wherein the specific binding partner is labelled with an electrophoretic, or zeta potential, label and the method further comprises, prior to step a), taking an initial measurement in an electric field of the velocity, displacement, zeta potential or electrophoretic mobility of any cells or other biological entities present in the sample and comparing the value obtained from this initial measurement with the value obtained in step c) such that any change in value is indicative of the presence of a cell or other target biological entity to which the specific binding partner is bound.

3. A method as claimed in claim 1 or claim 2, wherein at least two specific binding partners are used, one having an electrophoretic, or zeta potential, label and the other having a fluorescence label and wherein in step d) the observed value is indicative of the presence of a cell or other target biological entity to which both specific binding partners are bound.

4. A method as claimed in any of claims 1 to 3, wherein the specific binding partner is selected from the group consisting of: an antibody; a bacteriophage, a ligand for a receptor on the cell's surface; and an antibiotic.

5. A method as claimed in any preceding claim, wherein the label is a polyamino acid, a charged polysaccharide, a polynucleotide, a charged polymer or the like.

6. A method as claimed in any preceding claim, wherein the fluorescence label is selected from the group consisting of: acridine, AMCA, Alexa fluor 488 and 546, Bodipy labels, cascade blue, the Cy range of labels, or the like.

7. A method as claimed in any preceding claim, wherein the label on the specific binding partner is a positive zeta potential label whereby cells that become bound acquire a velocity in the opposite direction to that which they had before the binding reaction.

8. A method as claimed in any preceding claim, wherein the sample is first divided into two or more aliquots and then each aliquot is contacted with a different specific binding partner, allowing the binding partners to bind to any cells present in the respective aliquot, and measuring the velocity, displacement, zeta potential or electrophoretic mobility of each aliquot, whereby the pattern of changes in the values of the measured velocity, displacement, zeta potential or electrophoretic mobility with each of the two or more specific binding partners, forms a profile or fingerprint for the particular cell or cells present in the samples.

9. A method as claimed in any preceding claim, wherein the method is a method for the detection, speciation or determination of a micro-organism present in a sample.

10. A method as claimed in any preceding claim, wherein the method is a homogeneous assay method for detection or species, variant or strain determination of a micro-organism or detection or determination of any other molecular or cellular biological entity present in a sample using specific binding partners.

11. A method as claimed in claim 10, wherein the method is a homogeneous immunoassay assay method using specific antibodies.

12. A method as claimed in any preceding claim, wherein a plurality of specific binding partners are provided and contacted with the fluid sample, each having a different distinguishable zeta potential label and each specific binding partner having a specificity for a different target cell or other target biological entity whereby a multiplex assay may be carried out.

13. A method as claimed in any preceding claim, wherein a plurality of specific binding partners are provided and contacted with the fluid sample, each having a different distinguishable fluorescence label and each specific binding partner having a specificity for a different target cell or other target biological entity whereby a multiplex assay may be carried out.

14. A kit for use in the method of any preceding claim and comprising one or more specific binding partners for a target cell or other biological entity.

15. A kit according to claim 14 that additionally comprises one or more of the following: a container suitable for holding the sample; a buffering agent; and one or more containers each containing a zeta potential or fluorescence label for a specific binding partner.

16. A kit as claimed in claim 14 or 15 in which said one or more specific binding partners is/are each labelled with a respective zeta potential or fluorescence label.

17. A method for detecting the presence of cells or other target biological entities in a fluid sample, which method comprises:
a) in an electric field measuring the value of the velocity, displacement, zeta potential or electrophoretic mobility of any said cell or other target biological entity present in the fluid sample;
b) contacting the sample with a specific binding partner having an electrophoretic, or zeta potential, label;
c) allowing the specific binding partner to bind to any said cell or other target biological entity present in the fluid sample;
d) in an electric field measuring the value of the velocity, displacement, zeta potential or electrophoretic mobility of any said cell or other target biological entity present in the fluid sample again; and
e) comparing the values obtained in steps a) and d), whereby any change in those values is indicative of the presence of a cell or other target biological entity to which the specific binding partner is bound in the fluid sample.
